SEQUENCE LISTING (3 sequences)

SEQ ID NO: 1 - DNA, 1704 bp, mus musculus
atgaatctgattgggcactcccatttacaggctacggacgagttttctctttctgaaagcttatttggtatgctaacagaacacgcggcaggccctctggggcagaatctggatttggaatcgtactccccatacaacaatgtcccgtttcctcaagttcagccacagatttcctcctcgtcttactattccaacctgggcttctacccccaacaaccggaagactggtattctcctggcatctatgaactcaggcgaatgcccgctgagactgggtaccagggagagactgaggtatcagagatgcctgtgacaaagaagccgcgaatggccgcggcatcggcaggcagaataaaaggggatgagctgtgtgttgtctgtggagacagggcctctgggtaccactacaacgcgctcacctgtgagggctgcaaaggtttcttccgaagaagcattaccaagaacgccgtgtacaagtgtaagaacgggggcaactgcgtgatggacatgtacatgcgcaggaagtgccaggagtgccggctaaggaagtgcagagagatggggatgttggctgaatgtttgttaactgaaatccagtgtaaatctaaacggctaaggaaaaatgtgaagcagcacgctgatcagacagtgaatgaggacgacagcgaagggcgtgacttgcgacaagtgacctccacaaccaagttttgcagggagaaaacggaactcacggcagaccagcagaccctcctggattatattatggattcgtacaacaaacagagaatgcctcaggaaatcacaaataaaatcttaaaagaagaatttagtgcagaagaaaattttctcatattaacagaaatggcaaccagccatgtacagattctcgtagaattcacaaaaaagcttccagggtttcagacactggaccacgaagatcagattgctttgctcaaagggtccgcagtggaggccatgtttcttcgttcggcggagattttcaataagaaacttcctgccggacatgcagacctgttggaagaaagaattcgaaagagtggtatctctgatgagtatataaccccgatgttcagtttctataaaagtgttggagaactcaaaatgactcaggaggagtacgctctgctcacagcgatcgtcatcctctctccagacagacaatacatcaaggacagagaggcggtggagaagctgcaggagcccctgcttgatgtgctacaaaagctgtgcaagatgtaccagcctgagaacccacagcatttcgcctgcctcctgggtcgcctgacggaactccggacattcaaccatcaccacgctgagatgctgatgtcttggagagtgaatgatcacaagttcaccccgctcctctgtgagatctgggatgtgcagtgatggacaccagtggggctggctccttgtcctcctcggaacagaaaccttgtttcgtttgtacctggtttcactcaagaatctcaatgaatatttatgtggcaattatacacctcccacggttgtaaatacagactagatagaactgctttccccacactgtattttacaaggcttcaggaaaccccactggcatgcccttttggcctaattaaatcaattgttacttcaattctatctactgagctaggggcatattattcttcattcgacaatattatatatattttataa

SEQ ID NO: 2 - protein, 470 aa, mus musculus
MNLIGHSHLQATDEFSLSESLFGMLTEHAAGPLGQNLDLESYSPYNNVPFPQVQPQISSSSYYSNLGFYPQQPEDWYSPGIYELRRMPAETGYQGETEVSEMPVTKKPRMAAASAGRIKGDELCVVCGDRASGYHYNALTCEGCKGFFRRSITKNAVYKCKNGGNCVMDMYMRRKCQECRLRKCREMGMLAECLLTEIQCKSKRLRKNVKQHADQTVNEDDSEGRDLRQVTSTTKFCREKTELTADQQTLLDYIMDSYNKQRMPQEITNKILKEEFSAEENFLILTEMATSHVQILVEFTKKLPGFQTLDHEDQIALLKGSAVEAMFLRSAEIFNKKLPAGHADLLEERIRKSGISDEYITPMFSFYKSVGELKMTQEEYALLTAIVILSPDRQYIKDREAVEKLQEPLLDVLQKLCKMYQPENPQHFACLLGRLTELRTFNHHHAEMLMSWRVNDHKFTPLLCEIWDVQ

SEQ ID NO: 3 - DNA, 1413 bp, mus musculus
atgaacctaatcggacattcacacttacaagcaaccgatgaattctcactctcagagagcttatttggtatgctaacagaacacgcggcaggccctctggggcagaatctggatttggaatcgtactccccatacaacaatgtcccgtttcctcaagttcagccacagatttcctcctcgtcttactattccaacctgggcttctacccccaacaaccggaagactggtattctcctggcatctatgaactcaggcgaatgcccgctgagactgggtaccagggagagactgaggtatcagagatgcctgtgacaaagaagccgcgaatggccgcggcatcggcaggcagaataaaaggggatgagctgtgtgttgtctgtggagacagggcctctgggtaccactacaacgcgctcacctgtgagggctgcaaaggtttcttccgaagaagcattaccaagaacgccgtgtacaagtgtaagaacgggggcaactgcgtgatggacatgtacatgcgcaggaagtgccaggagtgccggctaaggaagtgcagagagatggggatgttggctgaatgtttgttaactgaaatccagtgtaaatctaaacggctaaggaaaaatgtgaagcagcacgctgatcagacagtgaatgaggacgacagcgaagggcgtgacttgcgacaagtgacctccacaaccaagttttgcagggagaaaacggaactcacggcagaccagcagaccctcctggattatattatggattcgtacaacaaacagagaatgcctcaggaaatcacaaataaaatcttaaaagaagaatttagtgcagaagaaaattttctcatattaacagaaatggcaaccagtcatgtacagattctcgtagaattcacaaaaaagcttccagggtttcagacactggaccacgaagatcagattgctttgctcaaagggtccgcagtggaggccatgtttcttcgttcggcggagattttcaataagaaacttcctgccggacatgcagacctgttggaagaaagaattcgaaagagtggtatctctgatgagtatataaccccgatgttcagtttctataaaagtgttggagaactcaaaatgactcaggaggagtacgctctgctcacagcgatcgtcatcctctctccagacagacaatacatcaaggacagagaggcggtggagaagctgcaggagcccctgcttgatgtgctacaaaagctgtgcaagatgtaccagcctgagaacccacagcatttcgcctgcctcctgggtcgcctgacggaactccggacattcaaccatcaccacgctgagatgctgatgtcttggagagtgaatgatcacaagttcaccccgctcctctgtgagatctgggatgtgcagtga

(19) United States
(12) Patent Application Publication
(10) Pub. No.: US 2004/0171018 A1
(43) Pub. Date: Sep. 2, 2004

(54) Mouse farnesoid x receptor sequences for use in comparative pharmacology

(76) Inventors: Jason A. Holt, Durham, NC (US); Jodi Marie Maglich, Durham, NC (US); John Tomlin Moore, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/483,637

(22) PCT Filed: Jul. 17, 2002
(86) PCT No.: PCT/US02/22621

Related U.S. Application Data
(62) Division of application No. 60/306,074, filed on Jul. 17, 2001.

(51) Int. Cl. C07K 14/705; C12Q 1/68; C07H 21/04

(52) U.S. Cl. 435/6; 435/69.1; 435/320.1; 435/325; 530/350; 536/23.5

(57) ABSTRACT

The present invention provides polynucleotides and polypeptides of the mouse Farnesoid X Receptor (FXR) as well as expression vectors and host cells for expression of the mouse FXR. Also provided are methods for screening for modulators of the mouse FXR and using these modulators in the treatment of FXR related disorders.

### FIELD OF THE INVENTION

The present invention relates to mouse Farnesoid X Receptor (FXR) nucleotide and polypeptide sequences and their use in comparative pharmacology.

### BACKGROUND OF THE INVENTION

Nuclear hormone receptors comprise a large superfamily of ligand-modulated transcription factors that, in part, mediate responses to steroids, retinoids, and thyroid hormones (for review see Beato et al., (1995) Cell 83: 851-857; Kastner et al., (1995) Cell 83: 859-869; Mangelsdorf and Evans, (1995) Cell 83: 841-850). Detailed analysis of the receptors, most notably the steroid class of receptors, has revealed multiple discrete functional modules within the family that display generalized functional characteristics (Tzukerman et al., (1994) Mol. Endocrinol. 8: 21-30). A variable amino-terminal domain, referred to as A/B, is present that typically contains a strong and autonomous activation function (AF1), shown to be critical for cell and target gene specificity (Tora et al., (1988) Nature 333: 677-684). A more carboxyl-terminal central region contains a DNA Binding Domain (DBD) characterized by two C4-type zinc fingers. The DBD binds to specific genomic response elements and thereby regulates the transcriptional activity of select genes containing the response elements. A Ligand Binding Domain (LBD) is present at the distal carboxyl terminus and contains a highly conserved second transactivation function (AF2) that is important for hormone- dependent transcriptional transactivation (Lanz and Rusconi, (1994) Endocrinology 135: 2183-2195). Sequences that function in nuclear localization, receptor dimerization, and interaction with heat-shock proteins (Gronemeyer and Laudet, (1995) CCQ 2: 1173-1308) are also present within the nuclear receptor substructure. Through the coordinated action of these separate functional domains, nuclear receptor activation by ligand culminates in modulation of target gene expression (Tsai and O=Malley, (1994) Ann. Rev. Biochem. 63: 451-486) and in certain cases, cross-talk with other cell signaling pathways such as the NF-kB (Stein and Yang, (1995) Mol. Cell. Biol. 15: 4971-4979) and AP-1 (Paech et al., (1998) Science 277: 1508-1510). Ultimately, ligand alters nuclear receptor function by altering the constellation of protein-protein interactions in which the receptor is engaged (for review, see Freedman, (1999) Cell 97: 5-8). The molecular details underlying the multitude of cellular effects mediated by nuclear receptors are the subject of intense research activity.

A key step towards progressing nuclear receptors as therapeutic targets is validating the utility of the receptors in non-human animal models. These studies require the isolation of relevant non-human orthologs that can then be used to carry out comparative pharmacology experiments.

The nuclear receptor Farnesoid X Receptor (FXR; Unified Nomenclature Committee designation NR1H4) has been shown to be regulated by endogenous bile acids such as chenodeoxycholate (Parks et al., (1999) Science 284, 1365-1367; Makishima et al., (1999) Science 284, 1362-1364; Wang et al., (1999) Mol. Cell 3, 543-553). In rodents, FXR has been shown to play an integral role in cholesterol and bile acid homeostasis by regulating cytochrome P450 7a (Cyp7a) expression. The enzyme encoded by this gene is a key target of cholesterol lowering therapies because CYP7A is the first and rate-limiting enzyme in the cholesterol degradation pathway (reviewed in Russell, D.W. (1999) Cell 97, 539-542; and Repa et al. (1999) Curr. Opinion Biotech. 10, 557-563). More recently, FXR has been shown to mediate its effect on the Cyp7a promoter via a regulatory cascade involving the nuclear receptors SHP-1 and LRH-1 (Goodwin et al., (2000) Mol. Cell 6, 517-526). Most preliminary studies providing insights into FXR biology have been performed in rodents.

However, assessing FXR compounds for target validation studies in a rodent model such as the mouse requires ligand characterization using the correct sequence of the full-length mouse FXR. Characterization of compounds using full-length mouse FXR assays will be necessary as well in extending the utility of FXR beyond bile acid and cholesterol homeostasis. Thus, comparative pharmacology using the correct sequence of full-length mouse FXR is an important goal in validating this receptor as a useful target in disease.

### SUMMARY OF THE INVENTION

A full-length mouse FXR sequence is provided. Also provided is a mouse FXR sequence, which has been modified to promote stability of the sequence in E. coli. The mouse FXR sequences of the present invention are useful as screening targets for the identification and development of FXR selective compounds. These agents are particularly useful in FXR comparative pharmacology.

Accordingly, an aspect of the present invention relates to an isolated mouse FXR polypeptide comprising the amino acid sequence of SEQ ID NO:2, or a variant of SEQ ID NO:2, or fragments thereof, which are regulated by endogenous bile acids or regulate bile acid homeostasis via interaction with cytochrome P450 7a (Cyp7a) expression thereby modulating the cholesterol degradation pathway.

Another aspect of the present invention relates to polynucleotides encoding a mouse FXR polypeptide of the invention wherein the polynucleotides comprise:

(a) a nucleic acid sequence of SEQ ID NO: 1 or 3 and sequences complementary thereto;

(b) a sequence which hybridizes under stringent conditions to a sequence as defined in (a);

(c) a sequence that is degenerate as a result of the genetic code to a sequence as defined in (a) or (b); or

(d) a sequence having at least 60%, more preferably 80%, identity to a sequence as defined in (a), (b) or (c).

Other related aspects of the present invention include expression vectors which comprise a polynucleotide of the invention and which are capable of expressing a polypeptide of the invention, host cells comprising an expression vector of the present invention, methods of producing a polypeptide of the invention which comprise maintaining a host cell of the invention under conditions suitable for obtaining expression of the polypeptide and isolating the polypeptide, antibodies specific for a polypeptide of the invention, and nonhuman transgenic animals expressing the mouse FXR or a mutant thereof.

The present invention also relates to methods for identification of a substance that modulates FXR activity and/or expression. In one embodiment, the method comprises contacting a polypeptide, polynucleotide, expression vector, host cell or nonhuman transgenic animal of the invention with a test substance and determining the effect of the test substance on the activity and/or expression of the polypeptide to determine whether the test substance modulates FXR activity and/or expression.

In addition, the present invention relates to compounds which modulate FXR expression or activity and which are identifiable by the methods referred to above.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The present invention relates to a mouse Farnesoid X receptor, referred to herein as FXR, and variants thereof. Sequence information for the mouse FXR is provided in SEQ ID NO: 1 (nucleotide and amino acid) and in SEQ ID NO: 2 (amino acid only). A mouse FXR sequence modified to be stable in E.coli, but which still encodes the FXR is depicted in SEQ ID NO:3. A polypeptide of the invention thus consists essentially of the amino acid sequence of SEQ ID NO: 2 or a variant of that sequence, or a fragment of either thereof.

The full-length FXR nucleic acid sequence depicted in SEQ ID NO:1 differs from any previously reported full-length mouse FXR cDNA sequences. This sequence was derived from mouse genome data (sequence from Accession number AA270183) and contains a novel 5′ region. The existence of the novel full-length contiguous sequence was confirmed by PCR amplification of the sequence from a mouse liver cDNA library.

When this sequence was cloned in E. coli vectors, however, the 5′ sequence was unstable and was deleted from the sequence by E. coli. Thus, also disclosed herein as SEQ ID NO:3 is a modified full-length mouse FXR sequence that codes for the same amino acid sequence but differs at 3′ nucleotide codon positions. The modified mouse FXR sequence of SEQ ID NO:3 is stable in E. coli and allows for the propagation and isolation of full-length FXR-containing plasmids. The difference in the mouse FXR sequences of the present invention versus previously reported mouse FXR sequences (GenBank Accession number U09416) is contained at the 5′ region of the cDNA that codes for the amino terminal domain of FXR. This region typically includes an AF-1 domain, a region generally required for biological activities.

Studies with an expression vector containing the full-length mouse clone of the present invention also revealed functional characteristics that differ from the previously reported mouse FXR full-length sequence (GenBank Accession number U09416). For example, in transient transfection assays in CV-1 cells, the full-length sequence of the present invention showed less dependence on exogenously added SRC-1 expression plasmid for full-activation by chenodeoxycholate than an analogous expression plasmid containing the previously reported full-length mouse FXR (GenBank Accession number U09416).

Accordingly, in one embodiment, the present invention relates to mouse FXR polypeptides in a substantially isolated or purified form. It will be understood that by “substantially isolated” the polypeptide may be mixed with carriers or diluents, which will not interfere with the intended purpose of the polypeptide and still be regarded as substantially isolated. A polypeptide of the invention can also be in a substantially purified form, in which case it will generally comprise the polypeptide in a preparation in which more than 50%, e.g. more than 80%, 90%, 95% or 99%, by weight of the polypeptide in the preparation is a polypeptide of the present invention. Routine methods, can be employed to purify and/or synthesize the proteins according to the invention. Such methods are well understood by persons skilled in the art, and include techniques such as those disclosed in Sambrook et al, Molecular Cloning: a Laboratory Manual, 2^{nd} Edition, CSH Laboratory Press, 1989, the disclosure of which is included herein in its entirety by way of reference.

Polypeptides of the invention consist essentially of the amino acid sequence of SEQ ID NO: 2, a variant of that sequence, or a fragment of either thereof.

By the term “variant” it is meant to be inclusive of polypeptides having a similar essential characteristic or basic biological functionality to the mouse FXR of SEQ ID NO:2. For example, essential characteristics of the mouse FXR include its regulation by endogenous bile acids such as chenodeoxycholate and its integral role in cholesterol and bile acid homeostasis via regulating cytochrome P450 7a (Cyp7a) expression thereby modulating the cholesterol degradation pathway. Mouse FXR mediates its effect on the Cyp7a promoter via a regulatory cascade involving the nuclear receptors SHP-1 and LRH-1 (Goodwin et al., (2000) Mol. Cell 6, 517-526). Preferably, a variant polypeptide is one that binds to the same ligands as FXR. Accordingly, in one embodiment of the present invention, like the mouse FXR of the present invention, the expression and/or activity of an FXR polypeptide variant is regulated by endogenous bile acids, while the FXR polypeptide variant regulates the expression and/or activity of cytochrome P450 7a (Cyp7a). A variant polypeptide having a same essential character as mouse FXR is identified by monitoring for one or more functions of the FXR such as the ability to regulate expression and/or activity of Cyp7a, the ability to interact with SHP-1 and/or LRH-1, or the ability of the polypeptide to be regulated by an endogenous bile acid such as chenodeoxycholate. A full-length variant polypeptide is preferably one that includes the entire FXR Ligand Binding Domain.

By “variant” as used herein it is also meant to be inclusive of FXR polypeptides that do not show the same activity as FXR but rather inhibit a basic function or activity of FXR. For example, a variant of the present invention may comprise a polypeptide which inhibits the ability of FXR to interact with SHP-1 and/or LRH-1 and to regulate expression and/or activity of Cyp7a, for example by binding to FXR or a ligand thereof to prevent activity mediated by ligand binding to FXR.

Typically, polypeptides with more than about 65% identity, preferably at least 80%, at least 85% or at least 90% and more preferably at least 95%, at least 97% or at least 99% identity, with the amino acid sequences of SEQ ID NO: 2, are considered as variants of the protein. Identity can be determined using a program such as a BLAST sequence alignment program. Such variants may include allelic variants and the deletion, modification or addition of single amino acids or groups of amino acids within the protein sequence, as long as the peptide maintains or inhibits a basic biological functionality of the FXR. Amino acid substitutions may be made, for example from 1, 2 or 3 to 10, 15, 20 or 30 substitutions. The modified polypeptide generally retains activity as a FXR. Conservative substitutions can be made, for example, according to the following Table 1. Amino acids in the same block in the second column and preferably in the same line in the third column may be substituted for each other.

TABLE 1

| | | |
|---|---|---|
| ALIPHATIC | Non-polar | G A P |
| | | I L V |
| | Polar- | C S T M |
| | uncharged | |
| | | N Q |
| | Polar-charged | D E |
| | | K R |
| AROMATIC | | H F W Y |

Exemplary substitutions are shown in Table 2.

TABLE 2

| Original Residue | Exemplary Substitutions |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Met; Leu; Tyr |
| Ser | Thr |
| Thr | Ser |
| Trp | Tyr |
| Tyr | Trp; Phe |
| Val | Ile; Leu |

Shorter polypeptide sequences, also referred to herein as fragments, are also within the scope of the invention. For example, a fragment of at least 15, 20 or 30 amino acids or up to 50, 60, 70, 80, 100, 150 or 200 amino acids in length is considered to fall within the scope of the invention as long as it either demonstrates a basic biological functionality of FXR or inhibits a basic functionality of FXR. In particular, but not exclusively, this aspect of the invention encompasses the situation when the protein is a fragment of the complete protein sequence and may represent particularly, the A/B domain (e.g., about amino acids 1 through 122 in the mouse FXR of SEQ ID NO:2), the DBD (e.g., about amino acids 123-187 in the mouse FXR of SEQ ID NO:2) or the LBD (e.g., about amino acids 188-468 of the mouse FXR of SEQ ID NO:2), alone or in combination. Such fragments can be used to construct chimeric receptors preferably with another nuclear receptor, more preferably with another member of the family of FXR nuclear receptors, or as intermediates in the production of full-length sequences. Such fragments of FXR or a variant thereof can also be used to raise anti-FXR antibodies. In this embodiment the fragment may comprise an epitope of the FXR polypeptide and may otherwise not demonstrate the ligand binding or other properties of FXR.

Polypeptides of the invention may be chemically modified, e.g. post-translationally modified. For example, they may be glycosylated or comprise modified amino acid residues. They may also be modified by the addition of histidine residues to assist in their purification or by the addition of a signal sequence to promote insertion into the cell membrane. Such modified polypeptides fall within the scope of the term “polypeptide” of the invention.

Polypeptides of this invention may also comprise fusion proteins wherein the FXR or a portion thereof, preferably the DNA binding or ligand binding domain of the FXR, is linked to a non-FXR-derived amino acid sequence. For example, the full length FXR can be linked with viral VP16 autonomous transactivation domain at the amino terminus to protein a constitutively active receptor. The fusion proteins can be expressed recombinantly in accordance with well-known methods via a host cell containing an expression vector for the fusion protein. Alternatively, these fusion proteins can be prepared synthetically using standard techniques.

The invention also includes nucleotide sequences that encode for FXR or variants thereof as well as nucleotide sequences that are complementary thereto. By “nucleotide sequence” or “polynucleotide” it is meant to be inclusive of RNA or DNA including genomic DNA, synthetic DNA or cDNA. Preferably the nucleotide sequence is a DNA sequence and most preferably, a cDNA sequence. Nucleotide sequence information is provided in SEQ ID NO: 1 and SEQ ID NO:3. Such nucleotides can be isolated from mouse cells or synthesized according to methods well known in the art, as described by way of example in Sambrook et al, 1989.

Typically a polynucleotide of the invention comprises a contiguous sequence of nucleotides which is capable of hybridizing under selective conditions to at least a portion of SEQ ID NO: 1 or SEQ ID NO:3.

The polynucleotide can be in single-, double- or triple-stranded form. In addition, polynucleotide, as used herein, can refer to triple-stranded regions comprising RNA or DNA or both. The strands in such regions may be from the same molecule or from different molecules. As used herein, the term polynucleotide also includes nucleic acids that contain one or more modified (e.g., tritylated) or unusual (e.g., inosine) bases. Thus, DNAs or RNAs with backbones modified for stability or for other reasons are also polynucleotides as that term is intended herein.

A polynucleotide of the invention can hybridize to SEQ ID NO: 1 or SEQ ID NO:3 at a level significantly above background. Background hybridization may occur, for example, because of other cDNAs present in a cDNA library. The signal level generated by the interaction between a polynucleotide of the invention and SEQ ID NO: 1 or SEQ ID NO:3 is typically at least 10-fold, preferably at least 100-fold, as intense as interactions between other polynucleotides and SEQ ID NO: 1 or SEQ ID NO:3. The intensity of interaction may be measured, for example, by radiolabeling the probe, e.g. with ³²P. Selective hybridization can typically be achieved using conditions of medium to high stringency. However, such hybridization may be carried out under any suitable conditions known in the art (see Sambrook et al, 1989). For example, if high stringency is required, suitable conditions include from 0.1 to 0.2×SSC at 60° C. up to 65° C. If lower stringency is required suitable conditions include 2×SSC at 60° C.

Polynucleotides of the present invention are also inclusive of modified sequences. For example, SEQ ID NO: 1 or SEQ ID NO:3 can be modified by nucleotide substitutions from 1, 2 or 3 to 10, 15, 25, 50 or 100 substitutions. The polynucleotide of SEQ ID NO: 1 or SEQ ID NO:3 may alternatively or additionally be modified by one or more insertions and/or deletions and/or by an extension at either or both ends. A polynucleotide of the present invention can also include one or more introns such as an intron from the genomic DNA for FXR. Additional sequences such as signal sequences that may assist in insertion of the polypeptide in a cell membrane can also be included. The modified polynucleotide generally encodes a polypeptide that has a FXR activity. Alternatively, a polynucleotide encodes a ligand-binding portion of a polypeptide or a polypeptide that inhibits FXR activity. Both degenerate amino acid substitutions and/or conservative amino acid substitutions can be introduced resulting in a modified sequence, for example as shown in the Table above.

A nucleotide sequence which is capable of selectively hybridizing to the complement of the DNA of SEQ ID NO: 1 or SEQ ID NO:3 will generally have at least 60%, at least 70%, at least 80%, at least 90%, at least 95%, at least 98% or at least 99% sequence identity to SEQ ID NO: 1 over a region of at least 20, preferably at least 30, for instance at least 40, at least 60, more preferably at least 100 contiguous nucleotides or most preferably over the full length of SEQ ID NO: 1 or SEQ ID NO:3.

Various computer programs for calculating identity and/or homology are available. For example, the UWGCG Package provides the BESTFIT program that can be used to calculate homology (for example used on its default settings) (Devereux et al.,(1984) Nucleic Acids Res. 12: 387-395). The PILEUP and BLAST algorithms can be used to calculate homology or line up sequences (typically on their default settings), for example as described in Altschul (1993) J. Mol. Evol. 36: 290-300; Altschul et al (1990) J. Mol. Biol. 215: 403-410.

Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence that either match or satisfy some positive-valued threshold score T when aligned with a word of the same length in a database sequence. T is referred to as the neighborhood word score threshold (Altschul et al, 1990). These initial neighborhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P (N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides, forms one aspect of the invention, as does a polynucleotide which has at least 95% sequence identity over 40 nucleotides.

The nucleotides according to the invention have utility in production of the polypeptides according to the invention, which may take place in vitro, in vivo or ex vivo. The nucleotides may be involved in recombinant protein synthesis or indeed as therapeutic agents in their own right, utilized in gene therapy techniques. Nucleotides complementary to those encoding FXR, or antisense sequences, may also be used in gene therapy.

Polynucleotides of the invention may be used as primers, e.g. PCR primers or primers for an alternative amplification reaction, or as probes, e.g. polynucleotides detectably labelled by conventional means using radioactive or non-radioactive labels. In addition, the polynucleotides may be cloned into vectors.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, or 700 nucleotides in length, or even up to a few nucleotides, such as five or ten nucleotides, short of SEQ ID NO: 1 or SEQ ID NO:3.

The polynucleotides of the present invention are also useful in the production of chimeric receptors or fusion proteins having a FXR component. In a preferred embodiment, the fusion protein comprises at least a DNA binding domain or a ligand binding domain of a mouse FXR fused with a non-FXR derived sequence. Non-FXR derived sequences can be selected so as to be suitable for the purpose to be served by the chimeric receptor. Examples of such sequences include, but are not limited to, glutathione-S-transferase, the DNA binding domain of yeast transcription factor GAL4 and other DNA binding domains such as the DNA binding domains for estrogen or glucocorticoid receptors, and the viral VP16 transcriptional activation domain. Chimeric receptors of the present invention may further comprise a detectable label such as a radioactive or fluorescent label. The chimeric receptors may also be bound to a solid support such as glass or plastic particles or plates or a filter.

The present invention also includes expression vectors that comprise nucleotide sequences encoding the polypeptides or variants thereof of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art and are taught in general references such as Sambrook et al. 1989.

Polynucleotides according to the invention may also be inserted into the vectors described above in an antisense orientation in order to provide for the production of antisense RNA. Antisense RNA or other antisense polynucleotides may also be produced by synthetic means. Such antisense polynucleotides may be used as test compounds in the assays of the invention to inhibit expression of the FXR. Such antisense agents may also be useful therapeutically to inhibit expression of the FXR gene.

Preferably, a polynucleotide of the invention, when used in a vector, is operably linked to a control sequence that is capable of providing for the expression of the coding sequence by the host cell, i.e. the vector is an expression vector. The term Aoperably linked≅ refers to a juxtaposition wherein the components described are in a relationship permitting them to function in their intended manner. A regulatory sequence, such as a promoter, Aoperably linked≅ to a coding sequence is positioned in such a way that expression of the coding sequence is achieved under conditions compatible with the regulatory sequence.

The vectors may be, for example, plasmid, virus or phage vectors provided with an origin of replication, optionally a promoter for the expression of the polynucleotide, and optionally a regulator of the promoter. The vectors may contain one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example in a method of gene therapy or in the production of nonhuman transgenic animals. Additional vector components known in the art are suitable for use in the present vectors and include, for example, processing sites such as a polyadenylation signal, ribosome binding sites, RNA splice sites, and transcriptional termination sequences.

Promoters and other expression regulation signals may be selected to be compatible with the host cell for which expression is designed. For example, yeast promoters include S. cerevisiae GAL4 and ADH promoters, S. pombe nmt1 and adh promoter. Viral promoters may also be used. Examples include, but are not limited to the Moloney murine leukemia virus long terminal repeat (MMLV LTR), the Rous Sarcoma virus (RSV) LTR promoter, the SV40 promoter, the human cytomegalovirus (CMV) IE promoter, adenovirus, HSV promoters (such as the HSV IE promoters), and HPV promoters, particularly the HPV upstream regulatory region (URR). Mammalian promoters include, but are not limited to, the metallothionein promoter that can be induced in response to heavy metals such as cadmium and β-actin promoters. Tissue-specific promoters are especially preferred. All these promoters are readily available in the art.

The vector may further include sequences flanking the polynucleotide giving rise to polynucleotides which comprise sequences homologous to eukaryotic genomic sequences, preferably mammalian genomic sequences, or viral genomic sequences. This will allow the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Examples of suitable viral vectors include, but are not limited to, herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide giving rise to the polynucleotide in the host genome. Replication-defective adenovirus vectors by contrast remain episomal and therefore allow transient expression.

The invention also includes cells that have been modified to express the mouse FXR polypeptide or variant or fragments thereof. Such cells include transient, or preferably stable higher eukaryotic cell lines, such as mammalian cells or insect cells, using for example a baculovirus expression system, lower eukaryotic cells such as yeast, or prokaryotic cells such as bacterial cells. Particular examples of cells which may be modified by insertion of vectors encoding for a polypeptide according to the invention include, but are not limited to, mammalian HEK293T, CHO, HeLa and COS cells. A polypeptide of the invention can also be expressed in cells of a transgenic non-human animal. Accordingly, transgenic non-human animals expressing a FXR polypeptide of the invention are also included within the scope of the invention. For example, a nonhuman transgenic animal other than a mouse can be generated that expresses a FXR of the present invention as well as its endogenous FXR gene. Mice can also be generated in which the endogenous FXR gene is knocked out and then replaced by a variant FXR polynucleotide of the present invention. Nonhuman transgenic animals can also be generated that express isoforms of FXR as well as mutant alleles of the FXR of the present invention. Transgenic animals developed by these methods can be used to screen compounds for drug interactions and toxicities and to study the regulation of FXR in vivo.

The present invention also relates to antibodies, specific for a polypeptide of the invention. Such antibodies are useful in a variety of procedures including, but not limited to, purification, isolation or screening methods involving immunoprecipitation techniques or, indeed, as therapeutic agents in their own right.

Antibodies may be raised against specific epitopes of the polypeptides according to the invention. Such antibodies may be used to block ligand binding to the receptor. An antibody, or other compound, specifically binds to a protein when it binds with preferential or high affinity to the protein for which it is specific but does not substantially bind or binds with only low affinity to other proteins. A variety of protocols for competitive binding or immunoradiometric assays to determine the specific binding capability of an antibody are well known in the art (see for example Maddox et al, (1993) J. Exp. Med. 158: 1211-1226). Such immunoassays typically involve the formation of complexes between the specific protein and its antibody and the measurement of complex formation.

Antibodies of the invention may be antibodies to mouse polypeptides or fragments thereof. For the purposes of this invention, the term antibody, unless specified to the contrary, includes fragments that bind a polypeptide of the invention. Such fragments include Fv, F(ab=) and F(ab=)₂ fragments, as well as single chain antibodies. Furthermore, the antibodies and fragment thereof may be chimeric antibodies, CDR-grafted antibodies or humanized antibodies.

Antibodies can be used in methods for detecting polypeptides of the invention in a biological sample. In these methods, a biological sample suspected of containing a polypeptide of the present invention is incubated with an antibody under conditions that allow for the formation of an antibody-antigen complex of the antibody and polypeptide. Formation of the antibody-antigen complex is then determined via well-known methods.

For purposes of the present invention, by “biological sample” it is meant to include, but is not limited to, tissue extracts, blood, serum, saliva, urine, cerebral spinal fluid, and bile.

Antibodies of the invention may be bound to a solid support and/or packaged into kits in a suitable container along with suitable reagents, controls, instructions, etc. Antibodies may be linked to a revealing label and thus may be suitable for use in methods of in vivo FXR imaging.

Antibodies of the invention can be produced by any suitable method. Means for preparing and characterizing antibodies are well known in the art, see for example Harlow and Lane (1988) AAntibodies: A Laboratory Manual≅, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the immunogen.

A method for producing a polyclonal antibody comprises immunizing a suitable host animal, for example an experimental animal, with the immunogen and isolating immunoglobulins from the animal=s serum. In this method, the animal is inoculated with the immunogen. Blood is subsequently removed from the animal and the IgG fraction purified.

A method for producing a monoclonal antibody comprises immortalizing cells that produce the desired antibody. Hybridoma cells can be produced by fusing spleen cells from an inoculated experimental animal with tumor cells (Kohler and Milstein (1975) Nature 256: 495-497). An immortalized cell producing the desired antibody may be selected by a conventional procedure. Hybridomas are then grown in culture or injected intraperitoneally for formation of ascites fluid or into the blood stream of an allogenic host or immunocompromised host.

For the production of both monoclonal and polyclonal antibodies, the experimental animal is suitably a goat, rabbit, or rat. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

An important aspect of the present invention is the use of polypeptides according to the invention in screening methods. The screening methods may be used to identify substances, particularly ligands, that bind to FXR. Screening methods may also be used to identify agonists or antagonists that may modulate FXR activity, inhibitors or activators of FXR activity, and/or agents which up-regulate or down-regulate FXR expression.

By the terms “modulate” or “modulating” it is meant an upregulation or downregulation in the level of expression of FXR and/or an increase or decrease in activity of the FXR.

Any suitable format may be used for the assay. In general terms such screening methods may involve contacting a polypeptide of the invention with a test substance and monitoring for binding of the test substance to the polypeptide or measuring receptor activity. A polypeptide of the invention may be incubated with a test substance. Modulation of FXR activity and/or expression may be determined by monitoring for changes in the interaction of FXR with endogenous bile acids such as chenodeoxycholate or changes in the level of Cyp7a. In a preferred aspect, the assay is a cell-based assay. Preferably the assay is carried out in a single well of a microtiter plate. Most preferred are assay formats that allow for high throughput screening.

Modulator activity can be determined by contacting cells expressing a polypeptide of the invention with a substance under investigation, also referred to herein as a test substance or test compound, and by monitoring an effect mediated by the test compound. The cells expressing the polypeptide may be in vitro or in vivo. The polypeptide of the invention may be naturally or recombinantly expressed. Preferably, the assay is carried out in vitro using cells expressing recombinant polypeptide. It is also preferred that control experiments be carried out on cells which do not express the polypeptide of the invention to establish whether the observed responses are the result of activation of the polypeptide.

For purposes of the present invention, by the term “effect” as used herein, it is meant that the presence of the test compound increases or decreases the binding capability, activity and/or expression levels of the FXR as compared to the binding capability, activity and/or expression of FXR in the absence of the test compound.

The binding of a test substance to a polypeptide of the invention can be determined directly. For example, a radiolabeled test substance can be incubated with the polypeptide of the invention and binding of the test substance to the polypeptide can be monitored. Typically, the radiolabeled test substance can be incubated with cell membranes containing the polypeptide until equilibrium is reached. The membranes can then be separated from a non-bound test substance and dissolved in scintillation fluid to allow the radioactive content to be determined by scintillation counting. Non-specific binding of the test substance may also be determined by repeating the experiment in the presence of a saturating concentration of a non-radioactive ligand.

Assays can be carried out using cells expressing FXR, and incubating such cells with the test substance optionally in the presence of a FXR ligand. Alternatively, an antibody capable of forming a complex with FXR can be used to mediate FXR activity. Test substances may then be added to assess the effect on such activity. Cells expressing FXR constitutively may be provided for use in assays for FXR function. Such constitutively expressed FXR may demonstrate FXR activity in the absence of ligand binding. Additional test substances may be introduced in any assay to look for inhibitors of ligand binding or inhibitors of FXR-mediated activity.

Assays may also be carried out to identify substances that modify, or more specifically up-regulate or down-regulate FXR expression. Such assays may be carried out for example using antibodies or other specific ligands for FXR to monitor levels of FXR expression. Other assays that can be used to monitor the effect of a test substance on FXR expression include using a reporter gene construct driven by an FXR regulatory sequence as the promoter sequence and monitoring for expression of the reporter polypeptide.

Assays can also be carried out using known ligands of other FXR to identify ligands that are specific for polypeptides of the invention. Assays comparing known FXR agonists or FXR antagonists to test compounds can also be performed.

Thus, the present invention also relates to methods of screening test compounds for their ability to interact with a FXR polypeptide of the invention. Preferably, the method comprises providing a polypeptide comprising the FXR LBD, and determining the ability of a test compound to interact therewith. Such a method can be used to identify a natural ligand for the FXR and to determine the suitability of a test compound for use as an agonist or antagonist of that receptor.

Screening assays of the present invention generally involve first determining the ability of a test compound to bind to the receptor (e.g., the LBD of the receptor). A compound that binds can then be tested for its ability to affect the activity of the receptor. By way of example, a FXR LBD-containing polypeptide of the invention can be coupled to a solid support, e.g., to plastic beads or plates, using well-known coupling agents. Test compounds (which can bear a detectable label) can then be contacted with the immobilized polypeptide and the interaction between the test compound and the polypeptide monitored.

In a preferred embodiment, the screening assay takes the form of a FRET (Fluorescence Resonance Emission Transfer assay (Nichols et al. (1998) Anal. Biochem. 257:112-119). This method comprises the steps of exposing a sample portion comprising the donor located at a first position and the acceptor located at the second position to light at a first wavelength capable of inducing a first electronic transition in the donor, wherein the donor comprises a complex of a lanthanide chelate and a lanthanide capable of binding the chelate and wherein the spectral overlap of the donor emission and acceptor absorption is sufficient to enable energy transfer from the donor to the acceptor as measured by detectable increase in acceptor luminescence, wherein the improvement comprises using a SRC-1 (LCD2, 677-696) lanthanide chelate. In particular, the lanthanide element may be Europium and the signal chelate may be Europium bound to FXR. The method can utilize a signal pair that is Europium bound to FXR and allophycocyanin (APC) bound to SRC-1 (see, e.g., Parks et al. (1999) Science 284:1365-1368).

Other interaction assays using the full length mouse FXR protein or the complete amino terminus of the mouse FXR, which can be routinely developed by one of skill in the art based upon teachings provided herein, include, but are not limited to, mammalian two-hybrid assays and identification of phage-display peptides that react with the amino terminus of the mouse FXR.

Suitable test substances for screening in the above assays include those derived from combinatorial libraries, defined chemical entities and compounds, peptide and peptide mimetics, oligonucleotides and natural product libraries, such as display (e.g. phage display libraries) and antibody products. In a preferred embodiment, the test substances comprise organic molecules, preferably small organic molecules that have a molecular weight of from 50 to 2500 daltons. Candidate test substances may comprise biomolecules including, saccharides, fatty acids, steroids, purines, pyrimidines, derivatives, structural analogs or combinations thereof. These test substances are obtained from a wide variety of sources including libraries of synthetic or natural compounds. In addition, known pharmacological agents may be subjected to directed or random chemical modifications, such as acylation, alkylation, esterification, amidification, etc. to produce structural analogs for screening.

Test substances may be used in an initial screen of, for example, 10 substances per reaction. Those test substances showing inhibition or activation are then tested individually. Test substances may be used at a concentration of from 1 nM to 1000 μM, preferably from 1 μM to 100 μM, more preferably from 1 μM to 10 μM. In a preferred embodiment, the activity of a test substance is compared to the activity shown by a known activator or inhibitor. A test substance that acts as an inhibitor may produce a 50% inhibition of activity of the receptor. Alternatively a test substance which acts as an activator may produce 50% of the maximal activity produced using a known activator.

Comparative pharmacology involves the use of a nonhuman animal model to either predict the effects of a compound in humans, or provide a contrast to the effects of a compound in humans. Accordingly, results from assays such as described above with the mouse FXR of the present invention provide important information with respect to whether a compound of interest modulates the receptor similarly (or differently) versus the analogous human receptor.

Thus, another aspect of the present invention is the use of test compounds that have been identified by screening techniques referred to above in mouse FXR in the treatment of disease states responsive to regulation of FXR activity/expression in humans. The treatment may be therapeutic or prophylactic. The condition of a patient suffering from such a disease state can thus be improved. In particular, such substances may be used in the modulation of bile acid synthesis and cholesterol and lipid homeostasis. Accordingly, disease states that may be treated are those linked to alterations in cholesterol metabolism or catabolism including, but not limited to, atherosclerosis, gall stone formation, and ischemic heart disease.

Substances identified as modulators of FXR expression or activity according to the screening methods outlined above may be formulated with standard pharmaceutically acceptable carriers and/or excipients as is routine in the pharmaceutical art. For example, a suitable modulator may be dissolved in physiological saline or water for injections. The exact nature of a formulation will depend upon several factors including the particular substance to be administered and the desired route of administration. Suitable types of formulation are described in detail in standard reference texts such as Remington's Pharmaceutical Sciences, Mack Publishing Company, Eastern Pennsylvania, 17^{th} Ed. 1985, the disclosure of which is included herein of its entirety by way of reference.

FXR modulators may be administered by enteral or parenteral routes such as via oral, buccal, anal, pulmonary, intravenous, intra-arterial, intramuscular, intraperitoneal, topical or other appropriate administration routes.

A therapeutically effective amount of a modulator is administered to a patient. The dose of a modulator may be determined according to various parameters, especially according to the substance used; the activity of the substance as determined by screening assays described herein; the age, weight and condition of the patient to be treated; the route of administration; and the required regimen. A physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

Polynucleotides encoding mouse FXR or a variant thereof that modulate FXR activity may also be used therapeutically. Polynucleotides, such as RNA or DNA, more preferably DNA, can be provided in the form of a vector and administered to a patient so that the mouse FXR or variant thereof is expressed in the patient.

Polynucleotides encoding the polypeptide may be administered by any available technique. For example, the polynucleotide may be introduced by needle injection, preferably intradermally, subcutaneously or intramuscularly. Alternatively, the polynucleotide may be delivered directly across the skin using a nucleic acid delivery device such as particle-mediated gene delivery. The polynucleotide may also be administered topically to the skin, or to mucosal surfaces, for example, by intranasal, oral, intravaginal or intrarectal administration.

Uptake of nucleic acid constructs may be enhanced by several known transfection techniques, for example, those including the use of transfection agents. Examples of these agents include cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 μg nucleic acid for particle mediated gene delivery and 10 μg to 1 mg for other routes.

Another aspect of the present invention relates to the use of polynucleotides encoding the mouse FXR polypeptides of the invention to identify mutations in FXR genes that may also occur in the human gene and may be implicated in human disorders. Identification of such mutations may be useful in the development of transgenic animals expressing a mutant gene. These transgenic animals may serve as useful models for human disorders. Identification of such mutations may also be used to assist in diagnosis or susceptibility to such disorders and in assessing the physiology of such disorders. Polynucleotides may also be used in hybridization studies to monitor for up- or down-regulation of FXR expression. Polynucleotides such as SEQ ID NO: 1 or SEQ ID NO:3 or fragments thereof may be used to identify allelic variants, genomic DNA and species variants.

The following nonlimiting examples are provided to further illustrate the present invention.

### EXAMPLES

### Example 1

### Characterization of the Sequence

The nucleotide and amino acid sequence of the full-length mouse FXR have been determined. These are set out in SEQ ID NOs: 1 and 2. The full-length mouse FXR sequence was identified by bioinformatic analysis of the mouse genome database using in silico sequence comparison tools (BLAST and HMMR). It was found that the coding region of the 5=protein differed from the published sequence. This new sequence was verified by re-sequencing the relevant portion of the mouse genome, and subsequently creating full-length mouse FXR expression constructs. Since the published mouse FXR sequence is unstable, multiple silent mutations were made to create the stable sequences of SEQ ID NO:1 and 3.

### Example 2

### Identification of FXR Modulators via a Cell Based Assay

The full length mouse FXR sequence of the present invention is used to develop cell based assays in a transient or stable format. For these assays, a receptor expression construct expressing the full length mouse FXR and a reporter construct composed of an FXR response element or elements linked to a minimal promoter and reporter gene is introduced into a cell line. A test compound is then added to the cell line at varying concentrations and the effect of the test compound on reporter gene expression is measured. A change in reporter gene expression in the presence of the test compound as compared to reporter gene expression in cells not exposed to the test compound is indicative of the test compound being a modulator of mouse FXR.

What is claimed is:

1. An isolated farnesoid X receptor polypeptide comprising:
(a) an amino acid sequence of SEQ ID NO:2;
(b) a variant of SEQ ID NO:2 which is regulated by endogenous bile acids or regulates bile acid homeostasis via interaction with cytochrome P450 7a (Cyp7a) expression thereby modulating the cholesterol degradation pathway; or
(c) a fragment of (a) or (b) which is regulated by endogenous bile acids or regulates bile acid homeostasis via interaction with cytochrome P450 7a (Cyp7a) expression thereby modulating the cholesterol degradation pathway.

2. The polypeptide according to claim 1 wherein the variant (b) has more than 80% identity to the amino acid sequence of SEQ ID NO:2.

3. A polynucleotide encoding a polypeptide according to claim 1.

4. The polynucleotide according to claim 3 which is a cDNA sequence.

5. A polynucleotide encoding a farnesoid X receptor which is regulated by endogenous bile acids or regulates bile acid homeostasis via interaction with cytochrome P450 7a (Cyp7a) expression thereby modulating the cholesterol degradation pathway, said polynucleotide comprising:
(a) a nucleic acid sequence comprising SEQ ID NO: 1 or SEQ ID NO:3 or a sequence complimentary thereto;
(b) a nucleic acid sequence which hybridizes under stringent conditions to the nucleic acid sequence as defined in (a);
(c) a sequence that is degenerate as a result of the genetic code to a sequence as defined in (a) or (b), but which still encodes a farnesoid X receptor polypeptide which is regulated by endogenous bile acids or regulates bile acid homeostasis via interaction with cytochrome P450 7a (Cyp7a) expression thereby modulating the cholesterol degradation pathway; or
(d) a sequence having at least 80% identity to a sequence as defined in (a), (b) or (c).

6. The polynucleotide of claim 3 wherein the polynucleotide encodes amino acids 188 through 486 set forth in SEQ ID NO:2.

7. The polynucleotide of claim 3 wherein the polynucleotide encodes amino acids 123 through 187 set forth in SEQ ID NO:2.

8. The polynucleotide of claim 3 wherein the polynucleotide encodes amino acids 1 through 122 set forth in SEQ ID NO:2.

9. A fusion protein comprising:
(a) a DNA binding or ligand binding domain of the farnesoid X receptor of claim 1; and
(b) a non-farnesoid X receptor-derived amino acid sequence.

10. An isolated polynucleotide encoding the fusion protein of claim 9.

11. An expression vector comprising a polynucleotide of claim 3.

12. A host cell comprising the expression vector according to claim 11.

13. An expression vector comprising a polynucleotide of claim 5.

14. A host cell comprising the expression vector of claim 13.

15. An expression vector comprising a polynucleotide of claim 10.

16. A host cell comprising the expression vector of claim 15.

17. An antibody specific for a polypeptide according to claim 1.

18. An antibody specific for a polypeptide of claim 2.

19. A method for the identification of modulators of farnesoid X receptor activity and/or expression comprising:(a) contacting a test substance and a mouse farnesoid X receptor polypeptide or polynucleotide; and
(b) determining an effect of the test substance on activity and/or expression of said polypeptide or polynucleotide.

20. A method according to claim 19 wherein the polypeptide is expressed in a cell.

21. A substance which modulates farnesoid receptor activity or expression identified in accordance with the method of claim 19.

22. A method of modulating bile acid synthesis and cholesterol and lipid homeostasis in a patient comprising administering to said patient an effective amount of a substance according to claim 21.

23. A method of treating a patient suffering from alterations in cholesterol metabolism and catabolism comprising administering to the patient an effective amount of a substance according to claim 21.

24. The method of claim 21 wherein the patient is suffering from atherosclerosis, gall stone formation, or ischemic heart disease.

25. A method of producing the farnesoid X receptor polypeptide according to claim 1 comprising maintaining a host cell under conditions suitable for obtaining expression of the polypeptide and isolating said polypeptide.
